(19) United States
(12) Patent Application Publication
(10) Pub. No.: US 2011/0047818 A1
(43) Pub. Date: Mar. 3, 2011

(54) Orthotic insert system

(76) Inventor: Rick A. Raimondo, Medford, NJ (US)

(21) Appl. No.: 12/584,019

(22) Filed: Aug. 31, 2009

(51) Int. Cl. A43B 13/38; A61F 5/14

(52) U.S. Cl. 36/43; 36/140

(57) ABSTRACT

An orthotic insert system which is configured to be inserted into footwear utilizes an orthotic member consisting of a posted orthotic element with a rear heel section and a forwardly extending arch support section tapered downwardly away from the rear heel section. The rear heel section of the posted orthotic element has first and second lateral walls of different heights and substantially flat upper and lower surfaces extending between the lateral walls. One end of the upper surface is the height of the first lateral wall and the other end of the upper surface is the height of the second lateral wall. The upper surface has a gradual slope as it extends from the first lateral wall to the second lateral wall. The orthotic member also has a rim which overlays the upper surface of the rear heel section. An insole member is positioned within the orthotic member. The orthotic member is not attached to the footwear nor is the insole member attached to the orthotic member. Proper fitting of the insole member within the orthotic member, which itself is fitted into the footwear, sets and maintains the angle of the heel, arch, and foot of a wearer correctly within the footwear.

### BACKGROUND OF THE INVENTION

Orthotic inserts placed in footwear are commonly used to provide foot support for the wearer, particularly where the wearer requires biomechanical support and control during standing, walking, and running. The orthotic insert is generally used to control excessive pronation and supination which is experienced when the foot strikes the ground during walking or running. Pronation is the flattening or rolling inward of the foot as the foot strikes the ground. Supination is the rolling outward of the foot during walking or running. If either of these motions become excessive, there can be damage to the back, hip, knees, ankles and feet. Posted orthotics, a type of orthotic insert, are prescribed by physicians to adjust or correct the angle of the wearer's heel in footwear, in order to prevent or correct the problems associated with pronation and supination.

Although posted orthotics are currently in use as an integral part of footwear, they are routinely custom made devices. Inherent in custom made products are quality issues, expense, and time to manufacture. Conversely, over the counter orthotics or non-custom devices, may not fit well or support the foot, thus limiting their effectiveness.

### SUMMARY OF THE INVENTION

It is thus an object of the present invention to provide an orthotic insert system which overcomes the limitations and disadvantages of existing orthotic devices.

It is an object of the present invention to provide an orthotic insert system which effectively adjusts and corrects the angle of the wearer's heel in a shoe at all times during standing, walking, and running.

It is another object of the present invention to provide an orthotic insert system which utilizes two separate components positioned, unattached, one within the other, to be inserted in footwear. Although they are not connected, motion between the components is eliminated.

It is a further object of the present invention to provide an orthotic insert system which utilizes an orthotic member comprising a separate independent posted orthotic element and a separate, independent insole member configured to be placed on top of and within the posted orthotic element.

It is still another object of the present invention to provide an orthotic insert system which permits the ready and easy replacement of one posted orthotic element for another, as per the requirements of the wearer.

It is another object of the present invention to provide an orthotic insert system which, when inserted into footwear, will not distract from the appearance of the footwear.

It is a further object of the present invention to provide an orthotic insert system which can be manufactured in various sizes, depending on the shoe size.

It is still another object of the present invention to provide an orthotic insert system which is economical to manufacturer and provides a viable alternative to expensive custom made orthotics.

These and other objects are accomplished by the present invention, an orthotic insert system configured to be positioned within footwear. The system utilizes an orthotic member consisting of a posted orthotic element and a rear heel section with or without a forwardly extending arch support section tapered downwardly away from the rear heel section. The rear heel section of the posted orthotic element has first and second lateral walls of different heights and substantially flat upper and lower surfaces extending between the lateral walls. One end of the upper surface is the height of the first lateral wall and the other end of the upper surface is the height of the second lateral wall. The upper surface has a gradual slope as it extends from the first lateral wall to the second lateral wall. The orthotic member also has a rim which overlays the upper surface of the rear heel section. An insole member is positioned within the orthotic member. The orthotic member is not attached to the footwear nor is the insole member attached to the orthotic member. The insole member may also have a rim for a better fit. Proper fitting of the insole member within the orthotic member, which itself is fitted into the footwear, sets and maintains the angle of the heel, arch, and foot of a wearer correctly within the footwear.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is an isometric view of the components of the orthotic system of the present invention in position in footwear.

FIG. 2 is an isometric exploded view of the components of the orthotic system of the present invention, and their relationship to the footwear in which they are utilized.

FIG. 3 is an isometric view of the orthotic member of the orthotic system of the present invention in position in footwear.

FIG. 4 is an elevation view of the orthotic member of the orthotic system of the present invention.

FIG. 5 is the other elevation view of the orthotic member of the orthotic system of the present invention.

FIG. 6 is a top view of the orthotic member of the orthotic system of the present invention.

FIG. 7 is a bottom view of the orthotic member of the orthotic system of the present invention.

FIG. 8 is a front view of the orthotic member of the orthotic system of the present invention.

FIG. 9 is a rear view of the orthotic member of the orthotic system of the present invention.

FIG. 10 is a cross-sectional view of the orthotic member of the present invention taken from FIG. 6.

FIG. 11 is a cross-sectional view of the orthotic member of the present invention taken from FIG. 6.

FIG. 12 is a cross-sectional view of the orthotic member of the present invention taken from FIG. 6.

FIG. 13 is a cross-sectional view of the orthotic member of the present invention taken from FIG. 6.

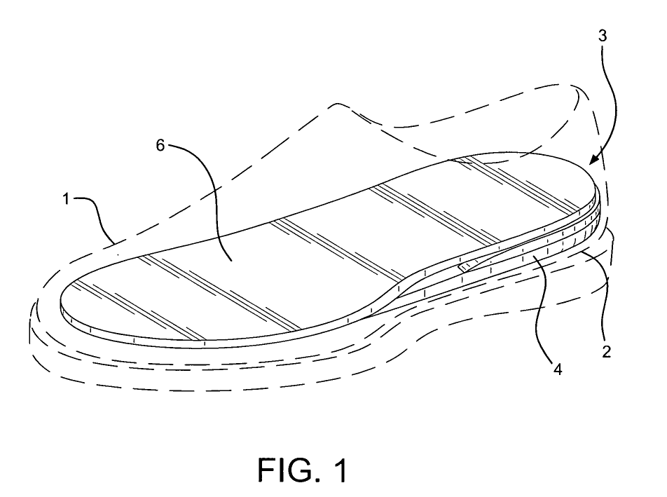

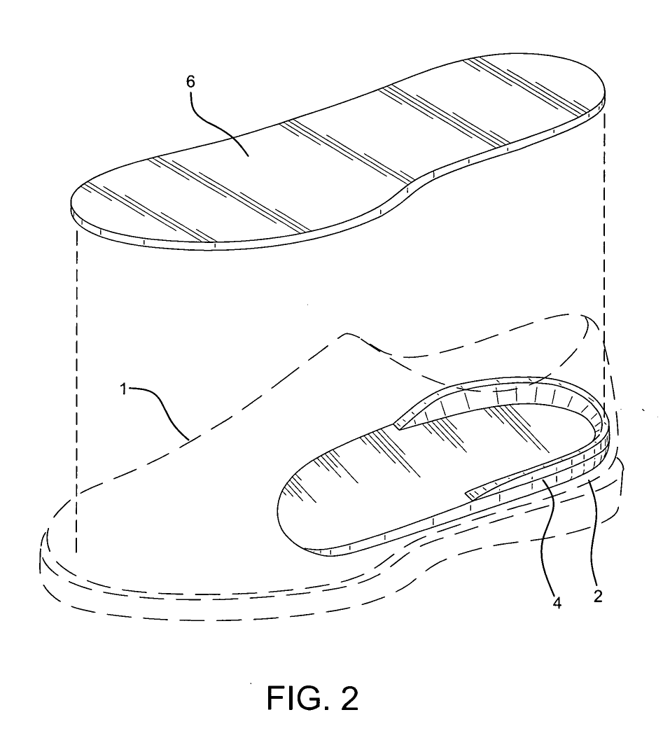

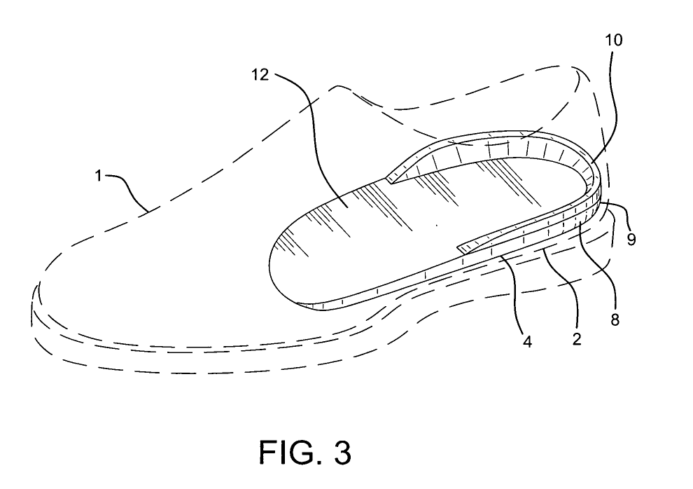

### DETAILED DESCRIPTION OF THE INVENTION

Orthotic system 3 of the present invention is designed to be positioned within footwear 1, having heel section 2 and a forwardly extending footwear base. Orthotic system 3 comprises orthotic member 4 which is to be fitted within heel section 2, and insole member 6 to be fitted within the orthotic member and to extend the length of footwear 1.

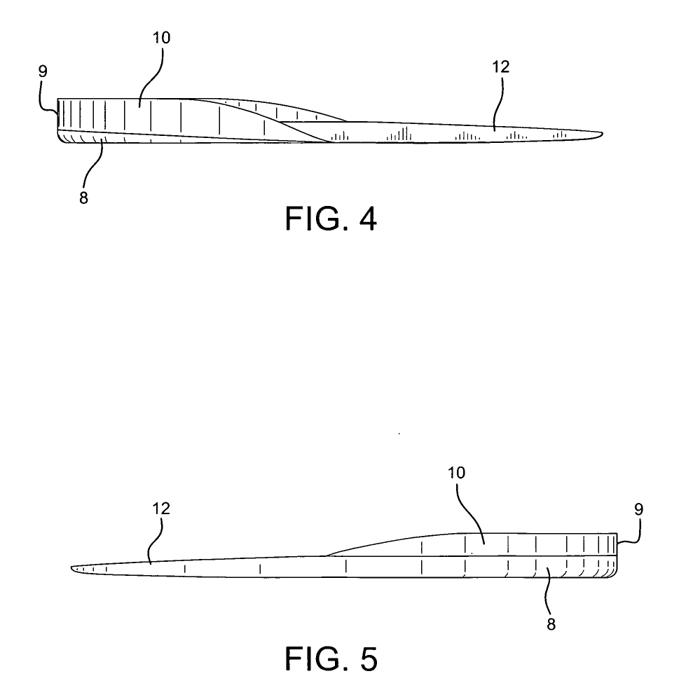

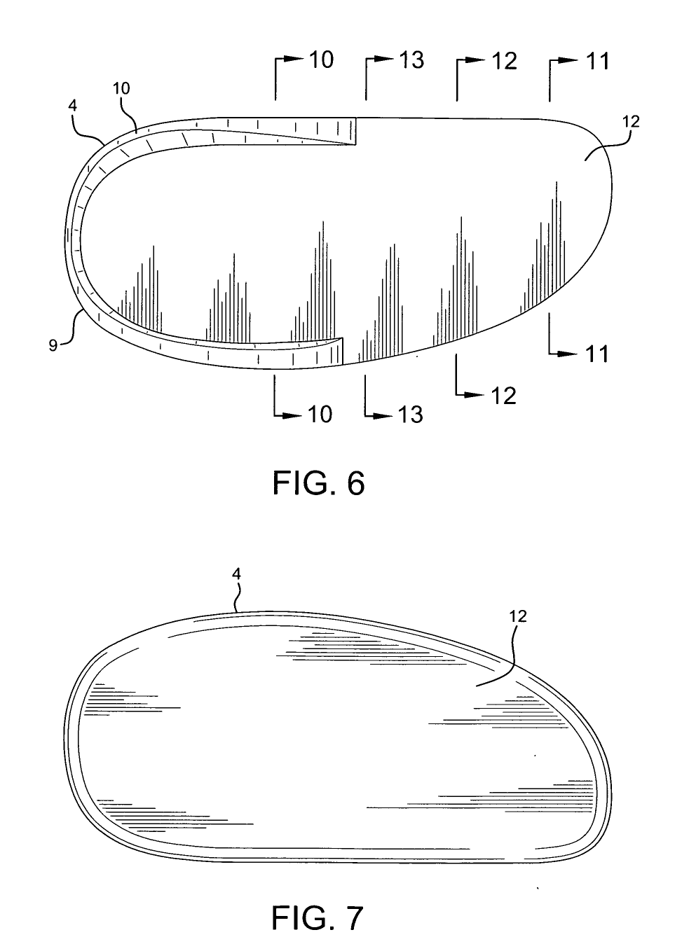

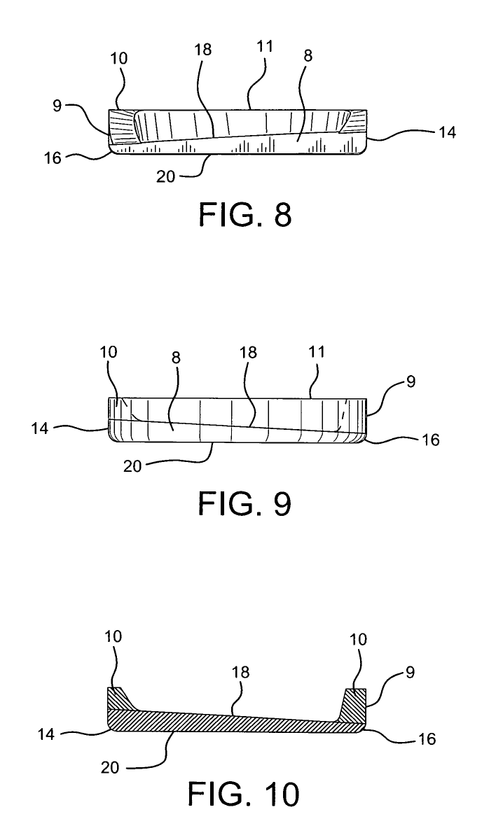

Orthotic member 4 comprises posted orthotic element 8 configured to be removeable positioned, without attachment, within heel section 2 of footwear 1. Posted orthotic element 8 comprises rear heel section 9 and arch support element 12. Rear heel section 9, as seen in FIGS. 4-6 and 8-10, is an asymmetrical element which is tapered as it extends around its heel shaped perimeter. Heel section 9 has first lateral wall 14 and second lateral wall 16 which is a lower height than the first lateral wall. Flat upper surface 18 extends between the lateral walls, such that one end of the upper surface is higher than its other end, as it gradually slopes downward from lateral wall 14 to lateral wall 16. Lower surface 20 of heel section 9 extends between lateral walls 14 and 16 with no taper. The height of lateral wall 14, i.e. the amount of posting or angular heel correction, can vary, depending on the footwear's requirement, but will generally range from ¼ inch to ⅜ inch.

Orthotic member 4 also comprises rim 10 which is positioned on top the outer perimeter of heel section 9 of posted orthotic element 8. Rim 10 is beveled downward to upper surface 18 posted orthotic element 8. Rim 10 has top surface 11 which is substantially parallel to lower surface 20 of posted orthotic element 8.

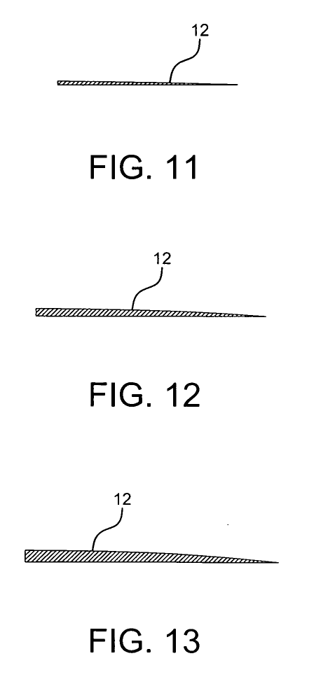

Finally, posted orthotic element 8 of orthotic member 4 comprises arch support section 12, which is tapered downward away from heel section 9, as best seen in FIGS. 11-13.

Orthotic system 3 further comprises insole member 6, which may be full or partial length to fit into most types of footwear. It is semi-rigid or cushioned in design. Insole member 6 may contain a raised border or lip around its perimeter and a variable amount of arch support. Most importantly, insole member 6 is contoured to fit on and within orthotic member 4.

In use, orthotic member 4 is positioned within footwear 1 such that heel section 9 of posted orthotic element 8 fits within heel section 2 of the footwear. Orthotic member 4 is not connected to footwear 1 in any manner. Insole member 6 is then positioned within orthotic member 4, such that the insole member fits in a snug manner without attachment, within rim 10 and in heel section 9 of posted orthotic element 8 and comfortably on arch support section 12 of the posted element. Both orthotic member 4 and insole member 6 are contoured to prevent motion between these two components, particularly with weight bearing. The profile of orthotic member 4 is low, such that it does not raise posted orthotic element 8 out of footwear 1. The wear's heel will rest on insole member 6, but the heel will tilt as per the slope of heel section 9 of posted orthotic element 8. Thus, by changing the angle of heel section 9, varying degrees of heel correction are achieved.

Moreover, the orthotic system of the invention serves to secure the position of insole member 6 within orthotic member 4 without the need for tape, adhesives, snaps or other attachment devices. The unique design of the components of the invention also permits easy and ready exchange of one orthotic member for another, as the need of the user requires. The overall length and width of orthotic member 4 and insole member 6 will vary, depending of footwear size.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. An orthotic insert system for use in footwear having a rear heel section and a forwardly extending base, said system comprising:
an orthotic member comprising a posted orthotic element configured to fit into the heel of, but not attached in any manner to, the footwear, said posted orthotic element having first and second lateral walls of different heights and substantially flat upper and lower surfaces extending between the lateral walls, one end of the upper surface being the height of first lateral wall and the other end of the upper surface being the height of the second lateral wall, the upper surface having a gradual slope as it extends from the first lateral wall to the second lateral wall, the orthotic member further comprising a rim overlaying the upper surface, said rim having a top surface which is substantially parallel to the lower surface of the posted orthotic element; and
an insole member positioned within, but not attached in any manner to, the orthotic member, whereby when the orthotic member is fitted into the rear heel section of footwear, the angle of the heel of a wearer is set and maintained correctly within the footwear.

2. The orthotic insert system as in claim 1 wherein the rim extends around the perimeter of the upper surface of the posted orthotic element.

3. The orthotic insert system as in claim 2 wherein the rim is beveled downward to the upper surface of the posted orthotic element.

4. The orthotic insert system as in claim 1 wherein the posted orthotic element further comprises a rear heel section and an arch support section tapered downward away from the rear heel section.

5. The orthotic insert system as in claim 1 wherein the insole member is contoured to fit within the orthotic member, whereby when the insole member is positioned therein, the insole member is secured in position in a snug manner within, but not attached to, the orthotic member and there is no movement therebetween when the footwear is being worn.

6. The orthotic insert system as in claim 1 wherein the orthotic member has a low profile, such that the orthotic element is not raised out of the footwear.
